(19) United States
(12) Plant Patent Application Publication
(10) Pub. No.: US 2017/0295688 P1
(43) Pub. Date: Oct. 12, 2017

(54) Lippia plant named 'ECOLOPIA2'

(50) ;

(71) Applicant: Masataka Ohde, Oyama (JP)

(72) Inventor: Masataka Ohde, Oyama (JP)

(21) Appl. No.: 14/999,180

(22) Filed: Apr. 8, 2016

(51) Int. Cl. A01H 5/02

(52) U.S. Cl. CPC A01H 5/02
USPC PLT/263.1

(57) ABSTRACT

A new and distinct Lippia cultivar named ‘ECOLOPIA2’ is disclosed, characterized by tolerance to humidity and disease, tall height and large leaf size. Flowers are uniquely light purple The new variety is a Lippia, useful for a variety of horticultural purposes.

Latin name of the genus and species: Lippia canescens×Lippia nodiflora.

Variety denomination:

‘ECOLOPIA2’.

### BACKGROUND OF THE INVENTION

The new Lippia cultivar is a product of a planned breeding program conducted by the inventor, Masataka, Ohde in Oyama, Japan. The objective of the breeding program was to produce new Lippia varieties for ornamental commercial applications. The cross resulting in this new variety was made during May 2008.

The seed parent is Lippia canescens ‘Campanga Verde’ U.S. Plant Pat. No. 20,120. The pollen parent is an unpatented Lippia nodiflora. The new variety was discovered on Jul. 1, 2010 by the inventor in a group of seedlings resulting from the 2008 crossing, in a research greenhouse in Oyama, Japan.

Asexual reproduction of the new cultivar ‘ECOLOPIA2’ was first performed at a commercial greenhouse in Oyama, Japan by terminal vegetative cuttings on Jun. 4, 2010. More than 3 generations have been reproduced by vegetative cuttings. Subsequently, propagation has also shown that the unique features of this cultivar are stable and reproduced true to type.

### SUMMARY OF THE INVENTION

The cultivar ‘ECOLOPIA2’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘ECOLOPIA2’. These characteristics in combination distinguish ‘ECOLOPIA2’ as a new and distinct Lippia cultivar:
1. Light purple flower color.
2. Tolerance to disease.
3. Tolerance to high humidity.
4. Above average height.
5. Above average leaf size.
6. Large inflorescence diameter.
7. Variety is sterile.

### PARENT COMPARISON

Plants of the new cultivar ‘ECOLOPIA2’ are similar to the pollen parent in most horticultural characteristics. The new variety however differs in the following characteristics:
1. Taller plant height, measured at foliar plane; the pollen parent grows to 4 cm, the new variety to 5 cm.
2. Leaves of the new variety are more rounded obovate.
3. The new variety has a better tolerance for disease.
4. Corolla color of ‘the pollen parent is Purple 76B, corolla color of ‘ECOLOPIA2’ is Purple 75B.

Plants of the new cultivar ‘ECOLOPIA2’ are similar to the seed parent, Lippia ‘Campagna Verde’ U.S. Plant Pat. No. 20,120 in most horticultural characteristics. The new variety however differs in the following characteristics:
1. Taller plant height, measured at foliar plane. ‘Campagna Verde’ grows to 3 cm, the new variety to 5 cm.
2. Larger inflorescence diameter: ‘Campagna Verde’ typically 0.7 cm, ‘ECOLOPIA2’ typically 1.3 cm.
3. Stronger tolerance to disease under high heat and high humidity conditions.
4. Corolla color of ‘Campagna Verde’ is Violet-Blue 91D, corolla color of ‘ECOLOPIA2’ is Purple 75B.
5. Leaf blade shape of ‘ECOLOPIA2’ leaf blade is more rounded obovate.

### COMMERCIAL COMPARISON

‘ECOLOPIA2’ can be compared to the unpatented, unnamed commercial Lippia nodiflora. Plants of Lippia nodiflora are similar to plants of ‘ECOLOPIA2’ in most horticultural characteristics. However ‘ECOLOPIA2’ differs from Lippia nodfflora in the following characteristics:
1. The new variety is observed sterile. The comparator produces many seeds.
2. Greater cold tolerance.
3. The new variety produces purple inflorescence, this comparator has white inflorescence.

### BRIEF DESCRIPTION OF THE PHOTOGRAPHS

The accompanying photograph in FIG. 1 illustrates in full color typical flowers and foliage of a plant of ‘ECOLOPIA2’ grown outdoors in Oyama, Japan.

FIG. 2 illustrates a rooted stem with inflorescence.

FIG. 3 illustrates a mass planting view of the plant grown outdoors.

FIG. 4 is a comparison of foliage. Foliage of ‘ECOLOPIA2’ is farthest left in the photo. The center leaf in the photo is the seed parent foliage, farthest right is the pollen parent foliage. Plants are typically grown as ground cover, the plants illustrated are planted directly in the ground. The photographs were taken using conventional techniques and although colors may appear different from actual colors due to light reflectance it is as accurate as possible by conventional photographic techniques.

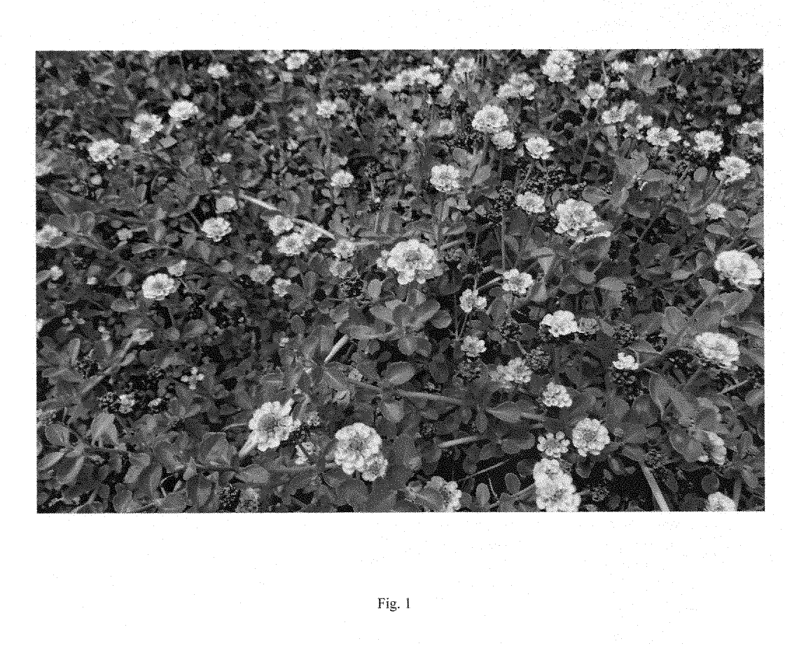

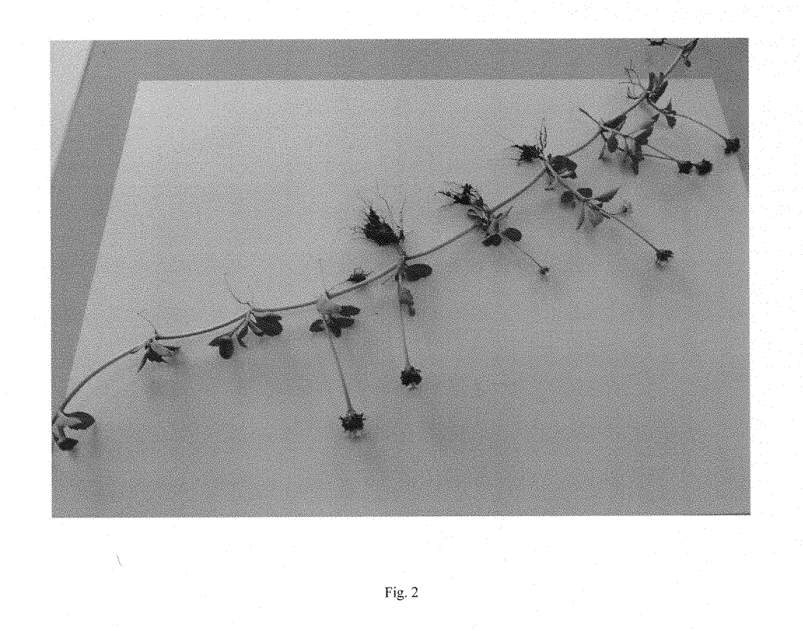

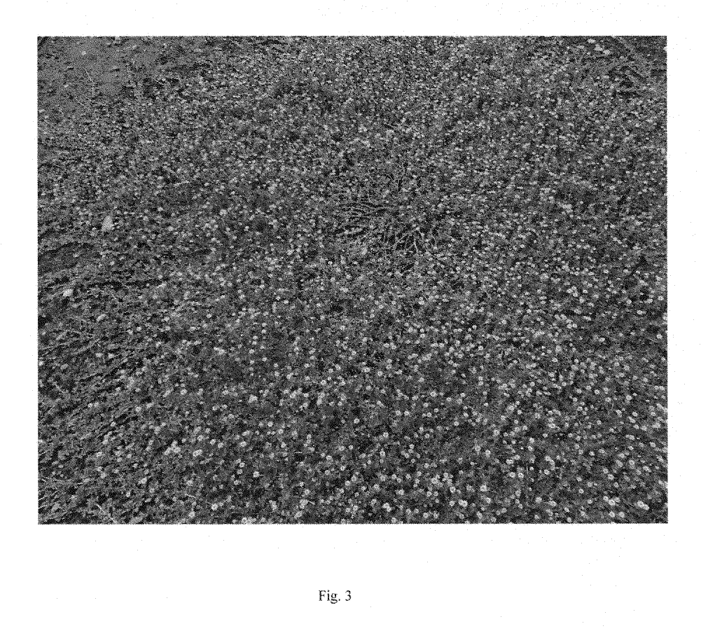

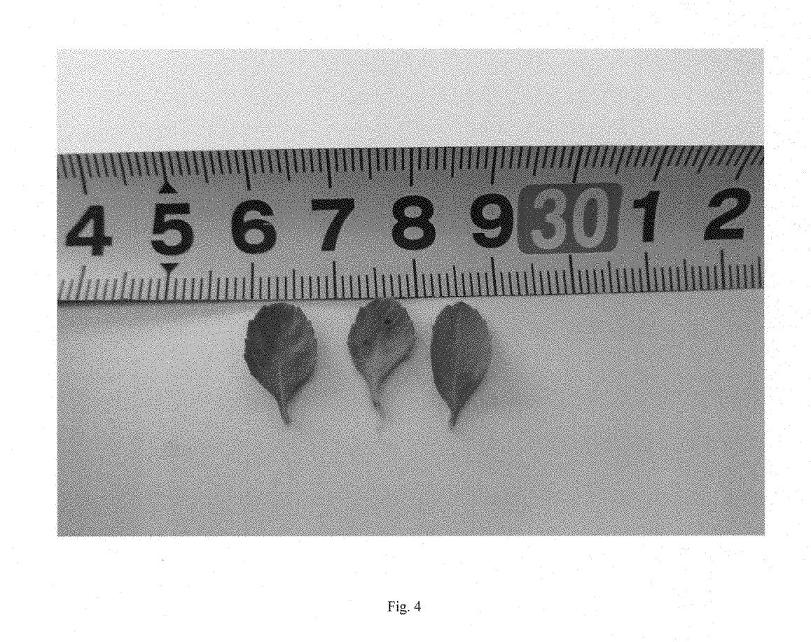

### DETAILED BOTANICAL DESCRIPTION

In the following description, color references are made to The Royal Horticultural Society Colour Chart 2007, except where general terms of ordinary dictionary significance are used. The following observations and measurements describe ‘ECOLOPIA2’ plants grown both outdoors and in a temperature controlled greenhouse in Oyama, Japan. Temperatures ranged from −7° C. to 25° C. at night to 3° C. to 50° C. during the day. No artificial light, photoperiodic treatments were given to the plants. Plants were grown both outside research field with plowed soil supplemented with fertilizer containing 8%N, 8%P, and 8%K, and in temperature-controlled and uncontrolled greenhouses in a mixture of Kanuma soil, peat and vermiculite supplemented with the same fertilizer. Measurements and numerical values represent averages of typical plant types.
Botanical classification: Lippia nodiflora ‘ECOLOPIA2’.
Propagation:
   Time to initiate roots.—3 to 7 days at approximately 23 ° C.
   Root description.—Thin root stretching to downward reaching as deep as 3 feet or deeper with sandy soil.
Plant:
   Age of plant described.—Approximately 90 days from rooted cutting.
   Time of year.—Jul. 2014.
   Growth habit.—Creeping, creating a dense mat.
   Planting description.—Not in a pot, planted in the ground outside of greenhouse.
   Height.—4 to 5 cm.
   Plant spread.—136 cm diameter, maximum in a year.
   Growth rate.—Vigorous.
   Branching characteristics.—Dense.
   Length of primary lateral branches.—68 cm.
   Diameter of lateral branches.—5 mm.
   Quantity of primary lateral branches.—Many, plant continuously forming branches.
   Characteristics of primary lateral branches.—Form: Round Diameter: 5 mm. Color: Near Green 137A, but darker. Some flushing Brown 200C Texture: Slightly scaly. Strength: Very strong, difficult to break.
   Internode length.—3-4 cm.
Foliage:
   Leaf.—Arrangement: Opposite Average Length: 1.2 cm. Average Width: 0.7 cm. Shape of blade: Very round obovate Apex: Broad acute Base: Attenuate Margin: Crenate Texture of top surface: Glabrous Texture of bottom surface: Glabrous Aspect: Slightly upwardly cupped Color: Young foliage upper(front) Near RHS Yellow-Green 146B Young foliage under(back) side: Near RHS Green 137D Mature foliage upper side: Near RHS Yellow-Green 146B Mature foliage under side: Near RHS Green 137D Venation: Type: Pinnate Venation color upper side: Near RHS Green 139D Venation color under side: Near RHS Green 139D Petiole: Leaves sessile.
Flower:
   Natural flowering season.—May to October.
   Days to flowering from rooted cutting.—1-2 months, depend on temperature and light conditions.
   Inflorescence and flower type and habit.—Terminal umbel.
   Persistent or self-cleaning.—Persistent.
   Bud.—Shape: Oval Length: 0.5 cm Diameter: 0.5 cm Color: Near RHS Purple 77A.
   Inflorescence.—Depth: 4.0 cm. Diameter: 1.8 cm Average quantity of individual flowers per inflorescence: 55.
   Corolla.—Individual flower: Depth: 0.5 cm Diameter: 0.3 cm Petals/lobes: Number: 1 Length: 0.5 cm Width: 0.3 cm Shape: Tube, flaring open at very end. Apex: Blunt round. Base: Fused Margin: Ruffled at apex. Texture: Glabrous all surfaces.
   Color.—When opening: Upper surface: Near RHS Purple 75B Lower surface: Near RHS Purple 75B Fully opened: Upper surface: Near RHS Purple 75B Lower surface: Near RHS Purple 75B 91D Throat: Color: Near RHS Purple 76D Texture: Glabrous. Tube: Interior Tube color: Near RHS Purple 75B, base near Yellow-Orange 17A Exterior Tube color: Near RHS Purple 75B Texture: Glabrous.
   Calyx.—Form Fused into a tube, Length: 0.2 cm Diameter: 0.2 cm Sepal texture: Glabrous Sepal color: Upper surface: Near RHS Green 139C Lower surface: Near RHS Green 139C.
   Fragrance.—None.
   Pedicels.—Not present.
   Peduncles.—Length: 3.0 cm to 5.0 cm Diameter: 0.1 cm Color: Near RHS Green 139C Texture: Glabrous.
Reproductive organs:
   Stamens.—Number (per flower): 4 Filament length: 0.05 cm Anthers: Shape: Linear Length: 0.05 cm Color: Near RHS Yellow-Orange 17A Pollen: Color: Near RHS Yellow-Orange 17B Amount: Scant.
   Pistils.—Quantity per flower: 1. Minute, not measurable. Color: Yellow-green (144A). Reproductive traits: Sex: Functional male (dioecious). Gynoecium: None. Androcoecium: Stamen number: 7. Anther shape: Narrowly deltoid. Anther attachment: Basifixed. Anther size: About 1 mm. Anther color: Green-white (157D). Amount of pollen: Abundant. Fruit and seed: Male selection. No fruit or seed present.
   DNA content.—2C DNA contend was determined following the methods of Shearer and Ranney (2013) using 4′, 6-diamidino-2-phenylindole (DAPI) fluorochrome stain and Pisum sativum ‘Ctirad’ as an internal standard with 3 replications. Mean 2C DNA content for ‘NCIV3’ was 4.27±0.07 (SEM) pg, approximately twice the value of I. verticillata ‘Southern Gentleman’ with a DNA content of 1.93±0.05 (SEM) pg indicating that ‘NCIV3’ is a tetraploid.
   Disease and insect resistance.—No significant disease or insect pests have been observed.
   Cold hardiness.—At least USDA zone 5; testing has not been completed in colder zones.
Comparison with commercial cultivars: Table 1 shows comparison of distinguishing characteristics between Ilex verticillata ‘NCIV3’, the female parent (NCSU 1998-538), and the commercial cultivar ‘Southern Gentleman’. ‘NCIV3’ is distinguished from Ilex verticillata ‘Southern Gentleman’ by having twice the genomic DNA content indicating it is a tetraploid and having a smaller, more compact stature.

TABLE 1
Comparison of Ilex verticillata taxa.

| | Taxa | | |
|---|---|---|---|
| | Ilex verticillata | | |
| | ‘Southern | Ilex verticillata | Ilex verticillata |
| Trait | Gentleman’ | NCSU 1998-538 | ‘NCIV3’ |
| Plant height & | About | About | About |
| diameter | 96 in. × 96 in. | 50 in. × 50 in. | 31 in. × 33 in. |
| Plant habit | Erect, | Rounded | Compact, |
| | rounded | | roughly |
| | shape | | rounded |
| | | | shape |
| DNA content (pg) | 1.93 ± 0.05 | 4.33 ± 0.05 | 4.27 ± 0.07 |
| mean ± SEM | | | |
| Estimated ploidy | Diploid. | Tetraploid | Tetraploid |
| level | | | |
| Sex | Male | Female | Male |

### Citations:

Shearer, K. and T. G. Ranney. 2013. Ploidy levels and relative genome sizes of species, hybrids, and cultivars of dogwood (Corunus spp.). HortScience 48(7):825-830.

### OTHER CHARACTERISTICS

Seeds and fruits: Not observed.

Disease/pest resistance: Excellent resistance to common disease and pests of Lippia nodiflora. New variety has better resistance to disease under humid and hot conditions than parents.

Temperature tolerance: Plant of the new hybrid have shown excellent resistance to high and low temperature extremes, having been grown successfully under temperature conditions ranging from a gentle frost outdoors, just below 0 degrees Celsius, to 50 degrees Celsius in an uncontrolled greenhouse.

Physical durability: New variety has excellent tolerance to be foot traffic.

Soil pH: New variety has excellent tolerance to a range of soil pH from 4.0-12.0

What is claimed is:

1. A new and distinct cultivar of Lippia plant named ‘ECOLOPIA2’ as herein illustrated and described.
